(19) United States
(12) Patent Application Publication
(10) Pub. No.: US 2015/0025552 A1
(43) Pub. Date: Jan. 22, 2015

(54) BRACE FOR TISSUE REPAIRS

(71) Applicant: Arthrex, Inc., Naples, FL (US)

(72) Inventor: Marc Stoll, Ft. Myers, FL (US)

(21) Appl. No.: 14/333,159

(22) Filed: Jul. 16, 2014

Related U.S. Application Data
(60) Provisional application No. 61/846,870, filed on Jul. 16, 2013.

(51) Int. Cl. A61F 2/00

(52) U.S. Cl. CPC A61F 2/0063
USPC 606/151

(57) ABSTRACT

Fixation of tissue such as bone or ligament is performed using a flexible material (for example, a suture net or brace or sling) with a portion having an expanded footprint to provide a sling or net or meshing for treatment of bone fractures, or for holding bone graft material and/or biologic material allowing precise placement of such bone grafts and/or biologic material at a repair site (for example, soft tissue or bone fracture repair site). The flexible material may have any shape and geometry that provides holding/supporting between the material and the tissue/bone to be fixated. The mesh or netting may be formed of a woven/braided/knitted/interlinked material (for example, a suture tape or any flexible material that can be knitted, woven, linked, braided, etc.).

### CROSS-REFERENCE TO RELATED APPLICATION

This application claims the benefit of U.S. Provisional Application No. 61/846,870 filed Jul. 16, 2013, the entire disclosure of which is incorporated by reference in its entirety herein.

### FIELD OF THE INVENTION

The present invention relates to the field of surgery and, in particular, to devices and techniques for reattachment and reinforcement of tissue such as bone, tendon or ligament.

### BACKGROUND OF THE INVENTION

Fracture healing is a proliferative physiological process in which the body facilitates the repair of a bone fracture. In the process of fracture healing, several phases of recovery facilitate the proliferation and protection of the areas surrounding fractures and dislocations. The length of the process depends on the extent of the injury, the angle of dislocation or fracture, and the extent of immobilization after the fracture surgery repair.

Ligaments and tendons are soft collagenous tissues that play a significant role in musculoskeletal biomechanics. Ligaments connect bone to bone and tendons connect muscles to bone. Repairing torn ligaments poses significant challenges for physicians. Some of the repairs include grafting tissue or placing artificial prostheses to replace ligaments. Often patients have to undergo multiple surgeries to maintain a repair, or have to have prostheses replaced.

Treatment of a torn ligament/tendon depends on the severity of the injury but often includes surgery with bracing and/or casting and immobilization. Bracing may cause complications such as stiffness or adhesions from limiting normal knee range of motion.

Successful and less invasive acute repairs that reduce progression to chronic instabilities and reconstruction procedures are needed. Also needed are devices and less invasive repairs for tissue reconstruction that improve primary repair outcomes without the cost of bracing, casting and/or delayed rehabilitation, for both fracture and soft tissue repairs. Systems and devices that are designed to hold bone grafts and other biologics, and then to further hold these bone grafts and biologics at a repair site (fracture, ligament, tendon repair site, etc.) are needed, as well as devices that reduce fracture fragments for comminuted fractures and that could be also used as an adjunct to ORIF (Open Reduction Internal Fixation) surgical procedures.

### SUMMARY OF THE INVENTION

The present invention provides minimally invasive techniques and devices for reattachment of tissue (such as bone or soft tissue) and reinforcing such reattached tissue with accelerated recovery and optimal length for bone/ligament healing.

The present invention provides a tissue net or brace and associated techniques according to which acute injury of tissue (for example, bone fracture or ligament repair) is treated with a reinforcement construct in the form of a mesh or netting of a woven/braided/knitted/interlinked material (for example, a suture tape or any flexible material that can be knitted, woven, linked, braided, etc.).

The reinforcement construct can be used as a netting to hold bone grafts and/or biologic material and to allow placement of such bone grafts and/or biologic material at a repair site (for example, soft tissue or bone fracture repair site). The reinforcement construct may be used as a sling to help reduce fracture fragments or may be used to provide a net to hold bone graft and augment fracture repair. The reinforcement construct (netting or FiberNet or sling) may be used to augment fracture repair of comminuted fractures, and may be employed as an aid in a fracture repair and care product line. The construct may be used as a sling to help reduce fracture segments or provide a simple net to hold bone graft. The reinforcement construct may be designed to be tied over bone or a button and/or incorporated into a bone plate, or used with any additional devices such as adjustable loop constructs (for example, adjustable knotless suture loop/button constructs, among many others). The reinforcement construct may be impregnated with biologics to aid in biological healing. An exemplary-only “sling” or FiberNet is designed to go around a bone and hold bone graft material and ACP to a fracture site.

The reinforcement construct acts as a “brace” that may be used for internal or external applications, eliminating the need of a post operative brace or cast in post-injury treatment, allowing immediate motion and rehabilitation, accelerating wound healing, and reinforcing the reattached soft tissue resulting in an early return to daily living and sports.

### BRIEF DESCRIPTION OF THE DRAWINGS

FIGS. 1 and 1(a) illustrate an exemplary flexible material of the reinforcement construct of the present invention (showing woven ultra-high molecular weight polyethylene (UHMWPE) core with a braided jacket of polyester and UHMWPE).

FIG. 2 illustrates a reinforcement construct in accordance with an exemplary embodiment of the present invention (a modular assembly option with a single high strength suture.

FIG. 3 illustrates a reinforcement construct in accordance with another exemplary embodiment of the present invention (a modular assembly option with two high strength sutures).

FIG. 4 illustrates a reinforcement construct in accordance with another exemplary embodiment of the present invention (a modular assembly option with two sutures and also with free sutures to help with positioning).

FIG. 5 illustrates a reinforcement construct in accordance with another exemplary embodiment of the present invention (a fixed option with three sutures).

FIG. 6 illustrates a reinforcement construct in accordance with another exemplary embodiment of the present invention (a fixed option with two swedged sutures).

FIGS. 7 and 7(a) illustrate a reinforcement construct in accordance with another exemplary embodiment of the present invention (a fixed option with loops which may be used with additional cinching loops or constructs).

FIG. 8 illustrates an exemplary proximal humerus fracture repair with a reinforcement construct of the present invention.

FIG. 9 illustrates an exemplary clavicle fracture (with plate and button) repair with a reinforcement construct of the present invention.

FIG. 10 illustrates an exemplary ankle repair (pilon fracture or periprosthetic fracture) with a reinforcement construct of the present invention.

### DETAILED DESCRIPTION OF THE INVENTION

The present invention provides a “tissue brace” or reconstruction system (FiberNet or sling) and associated techniques for treatment of acute injury of tissue (for example, bone or soft tissue repairs such as tendon or ligament repair).

The reconstruction system of the present invention comprises a flexible material in the form of a netting or mesh that may be optionally reinforced/assembled with additional flexible strands, cords or adjustable constructs (for example, additional sutures, high strength FiberTape® suture tape, high strength FiberWire® suture, TigerWire® suture, FiberChain® suture chain and/or FiberLink® loops, or adjustable suture loop/button constructs, among others) to form a resulting construct. The assembly options may be flexible/modular or fixed.

A biological material such as blood, blood components, BMA, BMSCs, PRP, ACP and/or growth factors may be injected into the injury site and small incisions and/or into the reinforcement construct, to further improve and accelerate incision and tissue healing. Alternatively, the reinforcement construct may be impregnated with such biological materials.

The reinforcement construct of the present invention may be employed with fixation devices (for example, screws or anchors such as SwiveLocks® or PushLocks®, or flexible adjustable knotted or knotless button/suture loop constructs) and/or with bone plates and fixation systems to simplify the overall surgical procedure and augment the ligament/tendon/bone repair, while reinforcing the reconnected ligament/tendon/bone. The reinforcement construct of the present invention may be used as a tissue protector/reinforcement construct.

The reinforcement construct of the present invention can be used as a netting to reduce fracture fragments for comminuted fractures, to hold bone graft and other biologics at a fracture site, or to be used as an adjunct to ORIF (Open Reduction Internal Fixation) procedures.

The reinforcement construct of the present invention may be also employed to augment fracture repair of comminuted fractures (as an adjunct to fracture care product lines) or can be used simply as a sling to help reduce fracture fragments or provide a net to hold bone grafts and/or biologics. The reinforcement construct may be tied over bone or a button and incorporated into a bone plate, for example. The reinforcement construct may be impregnated with biologics and may be used to aid in biological healing. A “sling” or FiberNet according to the present invention helps hold bone graft material and ACP to a fracture site. The reinforcement construct of the present invention may be permanently left at a repair site or may be removed.

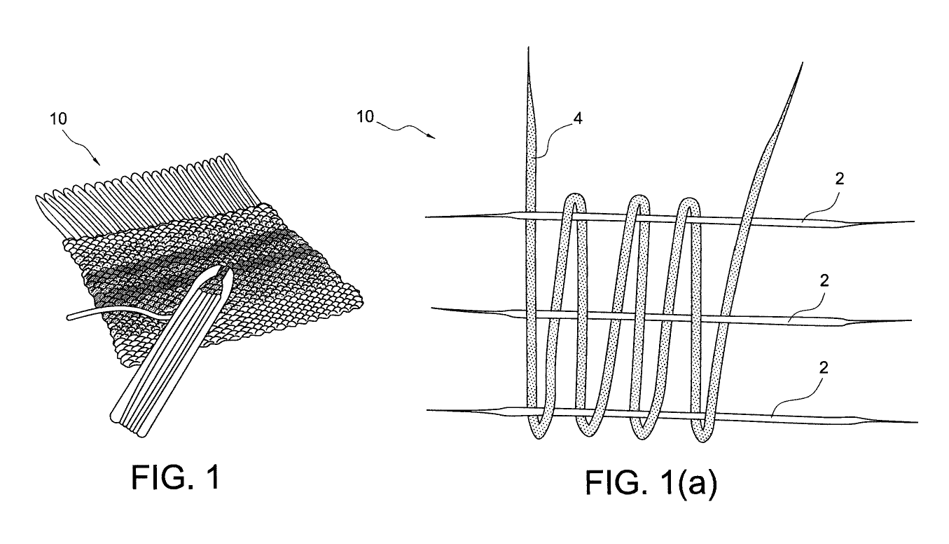

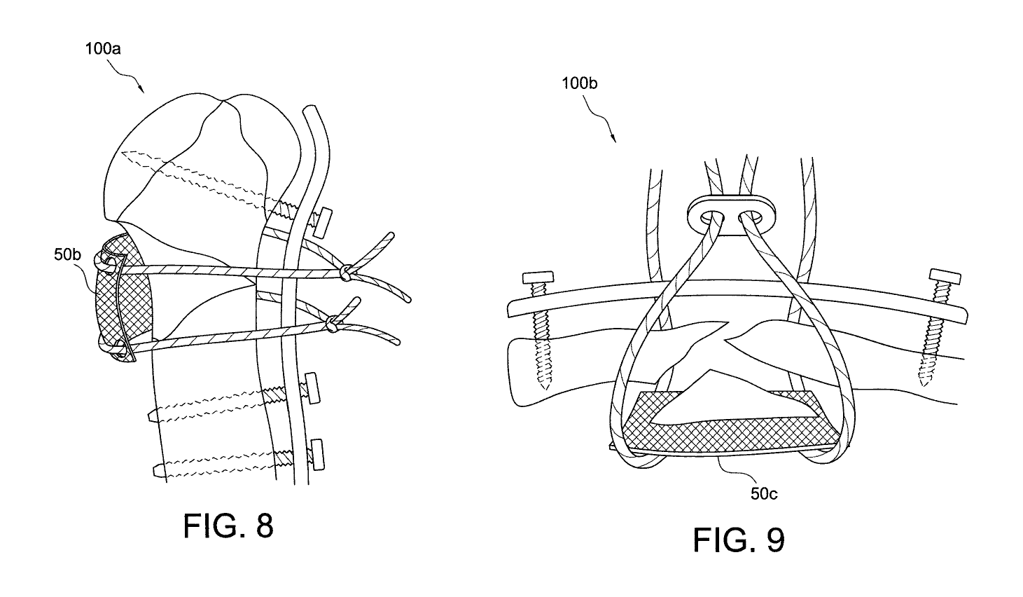

Referring now to the drawings, where like elements are designated by like reference numerals, FIGS. 1-7 illustrate exemplary embodiments of a tissue brace (or reconstruction system) 50a, 50b, 50c, 50d, 50e, 50f of the present invention, while FIGS. 8-10 illustrate exemplary tissue repairs 100a, 100b, 100c with the reconstruction systems of the present invention.

FIGS. 1 and 1(a) illustrate flexible material 10 in the form of an exemplary mat section 10 which may be any flexible material or combination of materials that can be woven, knitted, braided, interlocked, etc. In an exemplary-only and non-limiting embodiment, section 10 is formed of woven ultra-high molecular weight polyethylene (UHMWPE) core with a braided jacket of polyester and UHMWPE. In an exemplary embodiment only shown in FIG. 1(a), three flexible cords 2 (for example, three FiberWire® or TigerWire® sutures 2) are weaved/knitted/interlaced with additional strands 4 (for example, polyester or UHMWPE strands 4). The strands 2 and flexible material 10 may be manufactured from materials that have properties to amplify the body's healing response.

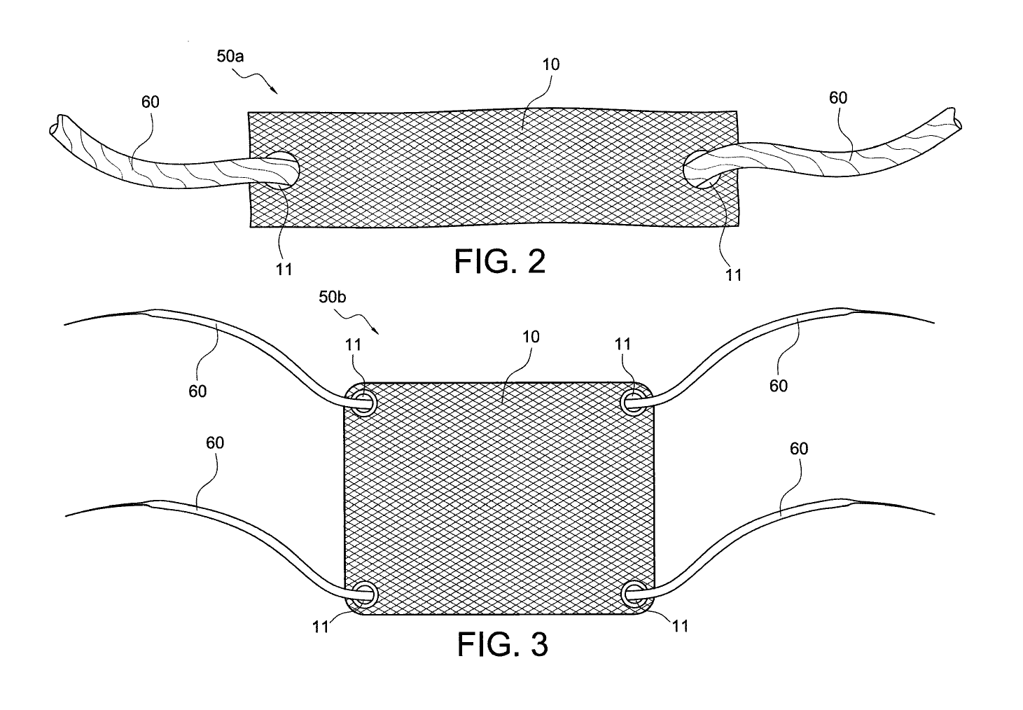

FIGS. 2-4 illustrate reconstruction systems 50a, 50b, 50c according to modular assembly options. In FIG. 2, flexible material 10 is provided with a single suture tape 60, for example, a FiberTape® 60 attached to (threaded through) the two ends of material 10. In an exemplary and non-limiting embodiment, the suture tape 60 is passed through end openings 11 located at the ends of the flexible material 10.

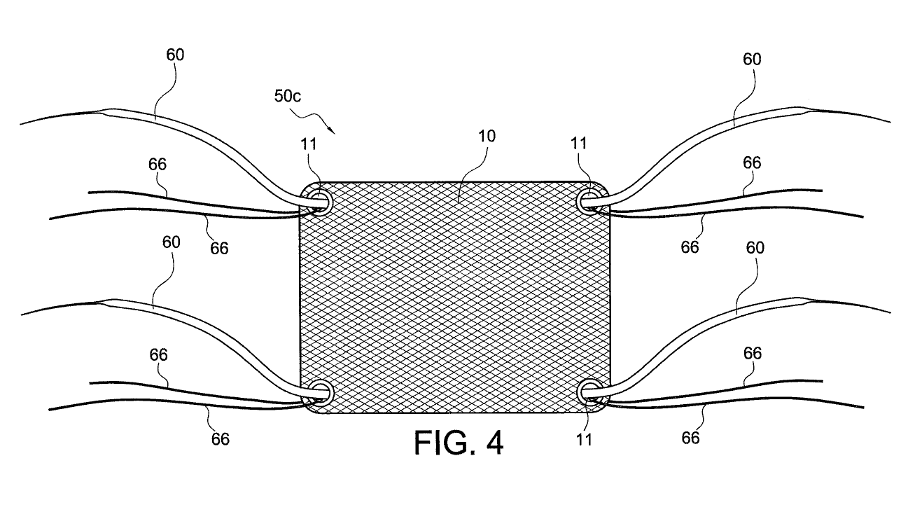

FIG. 3 illustrates reconstruction system 50b consisting essentially of flexible material 10 with two attached suture tapes, for example, two FiberTapes® 60 attached to (threaded through) the openings 11 at the ends of the material 10. FIG. 4 illustrates reconstruction system 50c consisting essentially of flexible material 10 with two attached suture tapes, for example, two FiberTapes® 60 attached to (threaded through) the openings 11 at the ends of the material 10, and also with additional flexible cords 66 (for example, free FiberWire® cords) that help with the positioning of the final construct.

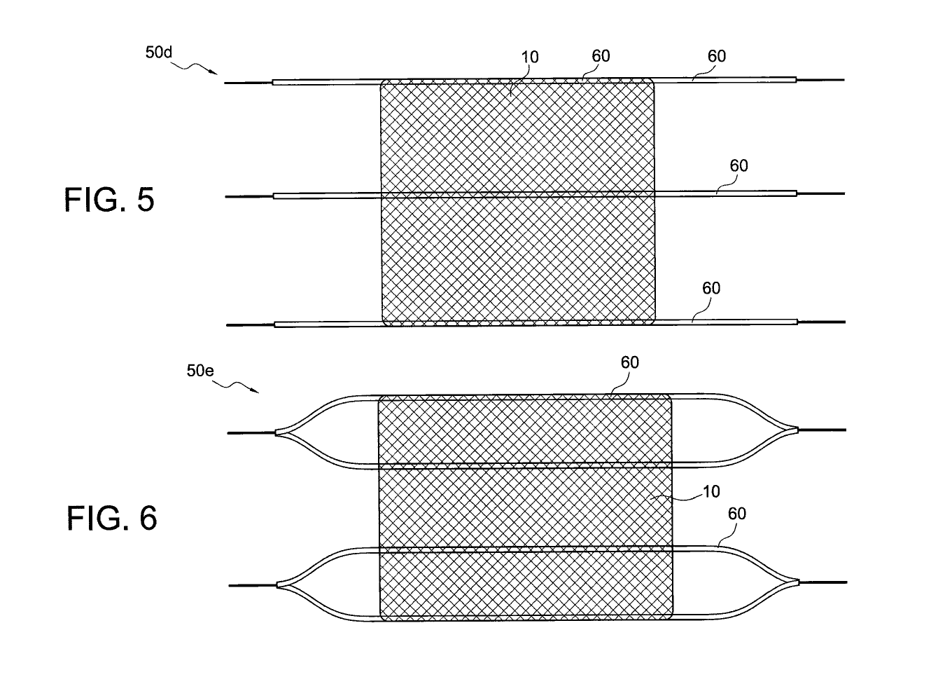

FIGS. 5-7 illustrate reconstruction system 50d, 50e, 50f according to fixed assembly options (i.e., the reinforcing tapes/strands are fixedly attached to the flexible material 10 and not sliding/passing through openings at the ends of the flexible material). FIG. 5 illustrates reconstruction system 50d consisting essentially of flexible material 10 with three securely-attached suture tapes, for example, three FiberTapes® 60 permanently affixed to the material 10.

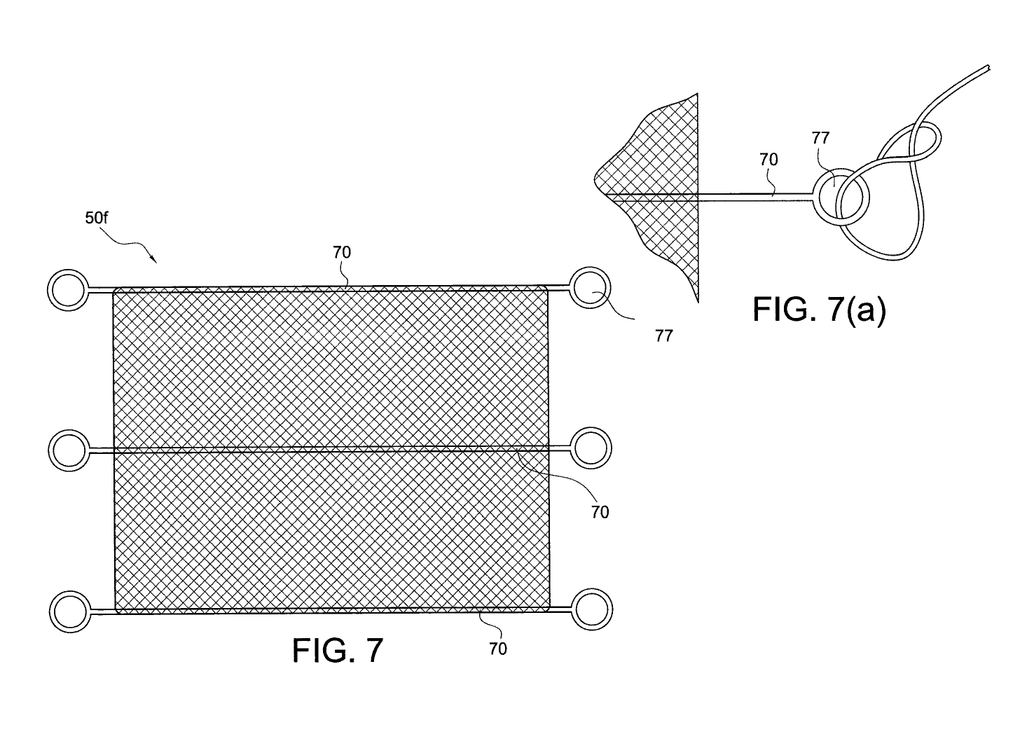

FIG. 6 illustrates reconstruction system 50e consisting essentially of flexible material 10 with two securely-attached suture tapes, for example, two swedged FiberTapes® 60 permanently affixed to the material 10. FIGS. 7 and 7(a) illustrate reconstruction system 50e consisting essentially of flexible material 10 with three securely-attached suture chains, for example, three FiberChains® 70 (with loops 77 used for FiberLink®) and permanently affixed to the material 10.

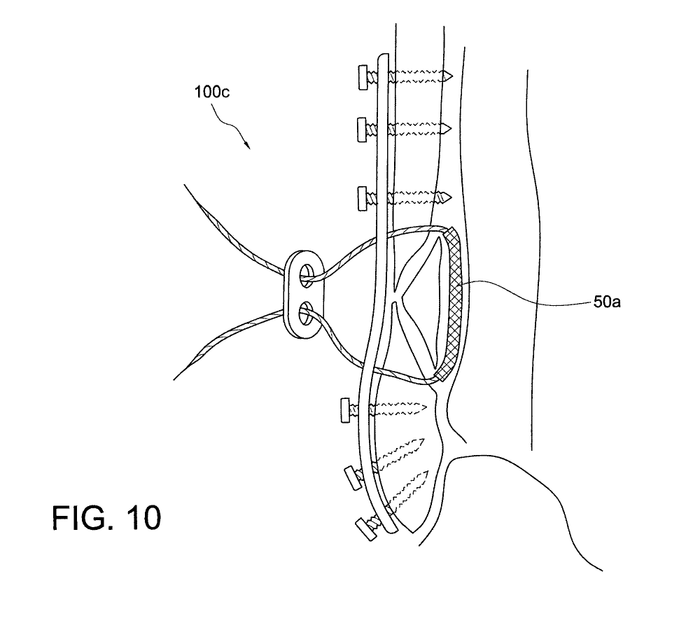

FIGS. 8-10 illustrate exemplary repairs 100a, 100b, 100c with the reconstruction systems of the present invention. FIG. 8 illustrates an exemplary proximal humerus fracture repair 100a with reinforcement construct 50b of the present invention. FIG. 9 illustrates an exemplary clavicle fracture repair 100b with plate and button repair and also with a reinforcement construct of the present invention, for example, with construct 50c. FIG. 10 illustrates an exemplary ankle repair 100c (pilon fracture or periprosthetic fracture) with reinforcement construct 50a of the present invention.

As noted above, at least one additional strand or flexible construct (suture, tape, chain, filaments, yarns, fibrils, loops, adjustable knotless suture/button constructs, etc.) may be added to/through the flexible material (in a fixed and/or non-fixed manner) to provide additional strength and support to the construct.

The materials employed for the formation of the reconstruction systems 50a, 50b, 50c, 50d, 50e, 50f may be loosely braided ultrahigh molecular weight polyethylene (UHMWPE) sutures, which may be braided with at least one other fiber, natural or synthetic, absorbable or resorbable, to form lengths of suture material. The reconstruction systems 50a, 50b, 50c, 50d, 50e, 50f may be also formed of suture tape such as FiberTape® as disclosed in U.S. Pat. No. 7,892,256, the disclosure of which is incorporated by reference in its entirety herewith.

The flexible material 10 may have various forms and geometries (for example, rectangular or square, among many others) that allow at least one addition element to attach thereto (either in a fixed or modular manner). As detailed above, the flexible material 10 of the constructs of the present invention may be formed of suture tape such as FiberTape® (disclosed in U.S. Pat. No. 7,892,256), suture chain such as FiberChain® (disclosed in U.S. Pat. No. 7,803,173), among others, or combination of these materials. Preferably, the flexible material may be a loosely braided UHMWPE suture that can be woven with additional strands/yarns/filaments to provide a braided/woven net (mesh) that can be used with additional reinforcing cords, strands and/or structures.

The reconstruction systems 50a, 50b, 50c, 50d, 50e, 50f may be woven or braided structures, or may be formed of yarns, fibers or similar materials, or combinations of these materials, that are joined/interlocked together by any known method in the art. In the exemplary-only embodiments above, the reconstruction systems 50a, 50b, 50c, 50d, 50e, 50f of the present invention are formed essentially of suture such as braided UHMWPE.

The reconstruction systems 50a, 50b, 50c, 50d, 50e, 50f could be utilized for multiple indications such as, for example, fracture repairs and ligament/tendon repairs such as AC joint reconstruction, syndesmosis reconstruction, quad/patellar tendon rupture repair, hallux-valgus repair, and any other tendon repair to bone. The reconstruction systems 50a, 50b, 50c, 50d, 50e, 50f detailed above may be also employed in conjunction with additional various knotted and/or knotless fixation devices (or combination of such knotted and knotless fixation devices), such as PushLock® anchors and/or SwiveLock® anchors to secure, for example, a medial row on rotator cuff repairs.

The reconstruction constructs and systems of the present invention may be also used in conjunction with any knotless fixation devices which can allow a flexible strand to be attached thereto. The fixation devices may be any of swivel and/or screw-in suture anchors and/or push-in suture anchors a knotless suture anchor such as the two-piece Arthrex PushLock® anchor, disclosed in U.S. Pat. No. 7,329,272, or an Arthrex SwiveLock® anchor, disclosed in U.S. Pat. No. 8,012,174 (the disclosures of which are herein incorporated by reference in their entireties). As detailed in these patents and applications, a knotless suture anchor is formed of an implant with a distal eyelet (which may be closed or forked, and which engages and secures at least one flexible strand) and a fixation device (a cannulated screw) that is advanced over the implant body to secure the implant and the flexible strand into a bone hole or socket, and to approximate soft tissue to bone. The implant may be rotatably attached to (and/or may swivel relative to) a tip of a driver assembly that is pre-loaded with the separate, cannulated fixation device (cannulated screw).

The fixation devices may be also any anchors, implants or screws (such as interference screws or tenodesis screws) or any fixation element that allows attachment/fixation of the constructs to bone. The fixation devices/implants may have various sizes (various diameters and/or lengths) and may be formed of biocompatible materials such as PEEK, biocomposite materials, metals and/or metal alloys, or combination of such materials, among others.

The flexible strands or sutures of the construct of the present invention may be also spliced—at least in part—in a manner similar to an Arthrex ACL TightRope®, such as disclosed in U.S. Pat. Nos. 8,439,976 and 8,460,379, the disclosures of which are incorporated by reference in their entirety herein.

Additional reinforcement constructs may be provided at a repair site (for example, fracture site) dependent upon the size of the tissue defect. Suture passing and knot tying are carried out in the preferred fashion to secure further attachment of soft tissue to bone, and if necessary.

The flexible strands employed for the formation of the reconstruction systems 50a, 50b, 50c, 50d, 50e, 50f may be high-strength sutures, such as the high strength suture sold by Arthrex, Inc. of Naples, Fla. under the registered tradename TigerWire® or FiberWire®, which is disclosed and claimed in U.S. Pat. No. 6,716,234, the entire disclosure of which is incorporated by reference in its entirety herewith. FiberWire® suture is formed of an advanced, high-strength fiber material, namely ultrahigh molecular weight polyethylene (UHMWPE), sold under the tradenames Spectra (Honeywell) and Dyneema (DSM), braided with at least one other fiber, natural or synthetic, to form lengths of suture material. The flexible strands may be also formed of suture tape such as FiberTape® as disclosed in U.S. Pat. No. 7,892,256, or a suture chain such as FiberChain® as disclosed in U.S. Pat. No. 7,803,173.

Biological materials such as blood, blood components, BMA, BMSCs, PRP, ACP and/or growth factors may be directly injected into the bone/ligament injury site and small incisions, to further improve and accelerate incision and tissue healing, or may be provided via the construct (i.e., provided within the flexible material 10 and/or additional cords/tapes, for example).

While the present invention is described herein with reference to illustrative embodiments for particular applications, it should be understood that the invention is not limited thereto. Those having ordinary skill in the art and access to the teachings provided herein will recognize additional modifications, applications, embodiments and substitution of equivalents all fall within the scope of the invention. Accordingly, the invention is not to be considered as limited by the foregoing description.

What is claimed is:

1. A tissue reinforcement construct, comprising:
a flexible material in the form of a mesh or netting; and
a plurality of flexible strands attached to the flexible material.

2. The tissue reinforcement construct of claim 1, wherein the flexible material is removably attached to the plurality of flexible strands.

3. The tissue reinforcement construct of claim 1, wherein the flexible material is permanently affixed to the plurality of flexible strands.

4. The tissue reinforcement construct of claim 1, wherein the flexible material is provided along a length of the plurality of flexible strands and supported by the flexible strands.

5. The tissue reinforcement construct of claim 1, wherein the flexible material comprises a plurality of eyelets, and wherein the plurality of flexible strands attach to the eyelets.

6. The tissue reinforcement construct of claim 1, wherein the flexible material is in the form of a woven ultra-high molecular weight polyethylene core with a braided jacket of polyester and ultra-high molecular weight polyethylene.

7. The tissue reinforcement construct of claim 1, wherein the flexible material is in the shape of a rectangle or square.

8. The tissue reinforcement construct of claim 1, wherein the plurality of flexible strands comprises at least one strand selected from the group consisting of suture, suture tape, suture cord, suture chain and nitinol strand.

9. The tissue reinforcement construct of claim 1, wherein the flexible material further comprises a biological component selected from the group consisting of platelet-rich plasma, autologous conditioned plasma and bone marrow aspirate.

10. The tissue reinforcement construct of claim 1, wherein the flexible material is a flat tape and the plurality of flexible strands consists of two swedged suture tapes.

11. The tissue reinforcement construct of claim 1, wherein the tissue is bone, ligament or soft tissue.

12. A reinforcement construct for a bone fracture, the reinforcement construct consisting essentially of a pad of flexible material provided along a length of a material strand.

13. The reinforcement construct of claim 12, wherein the pad is in the form of a rectangle or a square.

14. The reinforcement construct of claim 12, wherein the pad is provided along and around the length of material strand.

15. The reinforcement construct of claim 12, wherein the pad is formed of resorbable or non-resorbable material.

16. The reinforcement construct of claim 12, wherein the material strand is a suture, a nitinol strand, a yarn, a filament, a fiber strand, a suture tape, a suture tape or a suture chain.

17. The reinforcement construct of claim 12, wherein the pad is provided with a plurality of eyelets or openings.

18. The reinforcement construct of claim 12, wherein the pad is in the form of a woven ultra-high molecular weight polyethylene core with a braided jacket of polyester and ultra-high molecular weight polyethylene.

19. The reinforcement construct of claim 12, wherein the pad is positioned around the bone fracture to aid in supporting fractured bones of the bone fracture.

20. A method of reducing fracture segments of a fractured bone and holding bone graft at a fracture site, comprising the steps of:
providing a reinforcement construct comprising a flexible material in the form of a mesh or netting, and a plurality of flexible strands attached to the flexible material;
passing the flexible strands around the fractured bone so that the flexible material is positioned over and in contact with the fractured bone; and
securing the flexible material at the fracture site.

21. The method of claim 20, further comprising the step of providing bone graft material between the flexible material and the fractured bone, so that the bone graft material is held by the flexible material.

22. The method of claim 20, further comprising the step of providing ACP between the flexible material and the fractured bone.

23. The method of claim 20, wherein the fractured bone is humerus, clavicle or ankle.

24. The method of claim 20, further comprising the step of providing a biological material to the flexible material.

25. The method of claim 24, wherein the biological material is selected from the group consisting of blood, blood components, bone marrow aspirate, platelet rich plasma, autologous conditioned plasma, proteins, growth factors and hormones.

26. The method of claim 20, wherein the step of securing the flexible material at the fracture site further comprises tying at least one knot with the flexible strands over the fractured bone.

27. The method of claim 20, further comprising the step of attaching the flexible strands to a knotless fixation device by threading the flexible strands through an eyelet of the knotless fixation device, and then securing the knotless fixation device adjacent the fracture site.
